(19) United States
(12) Patent Application Publication
(10) Pub. No.: US 2018/0028416 A1
(43) Pub. Date: Feb. 1, 2018

(54) COSMETIC COMPOSITIONS WITH SENSORIAL AND AESTHETIC BENEFITS AND HAVING ENHANCED STABILITY

(71) Applicant: L'OREAL, Paris (FR)

(72) Inventors: Victoria Shin-wei FU, New York, NY (US); Catherine CHIOU, Saddle Brook, NJ (US); Lauren E. MANNING, Hoboken, NH (US)

(21) Appl. No.: 15/224,014

(22) Filed: Jul. 29, 2016

(51) Int. Cl. A61K 8/06; A61K 8/894; A61Q 17/04; A61K 8/81; A61K 8/58; A61K 8/891; A61K 8/895

(52) U.S. Cl. CPC A61K 8/064; A61K 8/891; A61K 8/894; A61K 8/895; A61K 8/8152; A61K 8/585; A61Q 17/04; A61K 2800/594

(57) ABSTRACT

Cosmetic compositions in the form of stable, pleasant feeling W/O or W/Si emulsions include an aqueous phase and an oil phase. The oil phase includes at least one silicone elastomer gel comprising at least one emulsifying elastomer, and at least one non-swelling non-emulsifying silicone elastomer. The composition also includes at least one stabilizing agent/spherical powder, water, and one or more additional components selected from actives and other additives. The compositions provide a smooth, non-greasy, silky feel when applied to the skin and other keratinous tissues, and impart to the skin a soft focus or hazy effect and the appearance of an even complexion.

### FIELD OF THE INVENTION

The present invention is directed to cosmetic compositions and the methods of use and production. More specifically, the present invention is directed to cosmetic compositions in the form of water-in-oil emulsions. The oil phase is a silicone elastomer gel that comprises of an emulsifying elastomer and at least one non-swelling, non-emulsifying silicone elastomer. Additionally, this emulsion also includes at least one stabilizing spherical powder and an aqueous phase comprising of water and one or more additional components selected from actives and other additives, as well as optionally comprises an aerogel.

### BACKGROUND OF THE INVENTION

Conventional cosmetic products, in particular for skin care, generally take the form of one or more actives that are solubilized, emulsified, or dispersed in a vehicle which is topically applied to the skin. A variety of emulsifiers may be used, and the form of the emulsion may be a water-in-oil or oil-in-water.

Emulsifying silicone elastomers are often selected because they provide aesthetic benefits to cosmetic formulations, particularly water-in-oil (W/O also referred to herein with respect to water-in-oil with silicone as W/Si) emulsions. Benefits include providing a smooth, non-greasy, silky feel when applied to the skin and other keratinous substrates such as hair and nails. Despite these benefits, W/Si emulsions generally suffer from poor stability. Indeed, in some examples, W/Si products are often thin, milky lotions that require shaking before application. It has been found that adding swelling non-emulsifying silicone gels to W/Si emulsions can thicken the oil phase and thus improve the stability of the emulsion, at least in the presence of certain actives. As an alternative to these swelling non-emulsifying silicone gels, non-swelling non-emulsifying silicone elastomer gels provide additional benefits and enhanced compatibility with some actives and conventional cosmetic additives; however, unlike the swelling non-emulsifying silicone gels, the non-swelling non-emulsifying silicone gels can unacceptably disrupt the stability of an emulsion, particularly under freeze/thaw (F/T) conditions.

In view of the various incompatibilities with swelling and non-swelling non-emulsifying silicone gels, there remains a need for formulations of W/Si emulsions that confer pleasant sensorial and aesthetic benefits to consumers and demonstrate stability.

### BRIEF SUMMARY OF THE INVENTION

In an exemplary embodiment, cosmetic compositions in the form of stable, pleasant feeling W/O and W/Si emulsions are provided. The compositions include an aqueous phase and an oil phase. The oil phase includes at least one silicone elastomer gel comprising at least one emulsifying silicone elastomer, and at least one non-swelling non-emulsifying silicone elastomer. The composition also includes at least one stabilizing agent/spherical powder, water, and one or more additional components selected from actives and other additives.

In another exemplary embodiment, cosmetic compositions include an oil phase comprising of silicone elastomer gel which contains at least one emulsifying silicone elastomer and at least one non-swelling non-emulsifying silicone elastomer, and a water phase comprising water and one or more additional components selected from actives and other additives. The composition also contains at least one stabilizing agent/spherical powder.

In another exemplary embodiment, cosmetic compositions comprise of an oil and a water phase. The oil phase makes up from about 10% to about 50% by weight of the total weight of the composition, the oil phase comprising of at least one silicone elastomer gel comprising at least one emulsifying silicone elastomer, and at least one non-swelling non-emulsifying silicone elastomer, the water phase is present at about 50% to about 90% by weight of the weight of the composition, the water phase comprising water, the composition further comprising at least one stabilizing agent/spherical powder, and, in various embodiments, one or more of the oil and the water phase comprising one or more additional components selected from actives and other additives.

In another exemplary embodiment, cosmetic compositions comprise of at least one stabilizing spherical powder present at from about 1% to about 15% by weight of the weight of the composition, at least one silicone elastomer gel comprising at least one of a water-in-oil (silicone) emulsifying elastomer present at from about 1% to about 20% by weight of the weight of the composition, and at least one of a non-swelling non-emulsifying silicone elastomer present at from about 1% to about 20% by weight of the weight of the composition. In various embodiments, the composition comprises one or more additional components selected from actives and other additives.

In another exemplary embodiment, a method for preparing the cosmetic compositions is provided involving mixing the above-disclosed ingredients to form the composition.

In accordance with the various embodiments, the cosmetic compositions are homogenous, water-in-oil emulsions that are stable, and provide a smooth, non-greasy, silky feel when applied to the skin and other keratinous tissues, and impart to the skin a soft focus or hazy effect and the appearance of an even complexion. In some embodiments, the compositions may comprise an aerogel. In some embodiments the compositions are devoid of and essentially lack at least one of an aerogel, a swelling emulsifying silicone elastomer, a swelling non-emulsifying silicone elastomer, and a swelling agent. In accordance with the various embodiments, the cosmetic compositions may comprise two or more of a silicone elastomer gel comprising at least one emulsifying elastomer, a non-swelling non-emulsifying silicone elastomer, a stabilizing agent/spherical powder, and combinations of these.

The present disclosure is also directed to a method for cosmetic treatment of keratinous tissues by applying the above-disclosed composition onto a surface of the keratinous tissue.

Other features and advantages of the present invention will be apparent from the following more detailed description of the preferred embodiment which illustrates, by way of example, the principles of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF THE INVENTION

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The term “comprising” (and its grammatical variations) as used herein is used in the inclusive sense of “having” or “including” and not in the exclusive sense of the terms “consisting only of,” “consisting essentially of” and “consisting of.”

“W/O emulsion,” and “W/Si emulsion” as used herein, includes a water phase dispersed in an oil phase, where the oil phase is a continuous phase and includes at least one Si emulsifier.

“Keratinous substrate,” as used herein, includes, but is not limited to, skin, hair, and nails. “Keratinous substrate” as used herein also includes “keratinous tissue” or “keratinous fibers,” which as defined herein, may be human keratinous fibers, and may be chosen from, for example, hair, such as hair on the human head, or hair comprising of eyelashes or hair on the body.

“Homogenous” means having the visual appearance of being substantially uniform throughout, i.e., visually appears as a single phase emulsion.

“Haze” refers to the diffusivity of a product, and its ability to re-direct rays of light passing through as determined in a conventional drawdown test.

“Transmission” relates the percentage of light that passes through drawdown without being absorbed or deflected by a product.

Applicants have surprisingly discovered that spherical polymer powders significantly enhance the stability of W/Si emulsions formed with non-swelling non-emulsifying silicone elastomer gels. This is particularly useful to meet the commercial requirements of shelf stability, especially through cycles of temperature variation, as evidenced in freeze/thaw (F/T) stability demonstration. This is a significant advancement in skincare to deliver stable cosmetics, for example stable SPF formulations, that provide a smooth, non-greasy, silky feel when applied to the skin and other keratinous tissues, and impart to the skin a soft focus or hazy effect and the appearance of an even complexion

An advantage of an embodiment of the present disclosure includes providing a W/O cosmetic composition for incorporating water soluble actives, particularly sunscreen actives. Another advantage of an embodiment of the present disclosure includes providing cosmetic compositions in a stable and aesthetically and sensorially pleasing form.

The W/O emulsion of the cosmetic composition according to the present disclosure has a stable, white, glossy cream appearance. When the cosmetic composition is applied to the skin in a conventional way, the cosmetic composition has a melting and caring sensation, with a velvet-soft skin finish.

Oil Phase

The oil phase present in the cosmetic composition, according to the disclosure, includes at least one silicone elastomer gel comprising an emulsifying crosslinked silicone elastomer having both silicone chain branches and alkyl chain branches swelled in a non-polar, volatile swelling agent, an organic sunscreen active, and optionally, a branched- and/or linear-type silicone emulsifier.

The oil phase cosmetic composition is at a concentration, by weight, of from about 10% to about 50%, and in some embodiments, from about 20% to about 40%, and in some embodiments, from about 35% to about 45%, based upon weight of the cosmetic composition. Thus, in various embodiments, the oil phase is present in a cosmetic composition according to the disclosure in a weight percent amount from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, to about 50 percent by weight, including increments and ranges there between.

Silicone Elastomer Gel

In accordance with the various embodiments, compositions according to the disclosure comprise at least one silicone elastomer gel, comprising, for example, crosslinked silicone elastomer having both silicone chain branches and alkyl chain branches is swelled in a non-polar, volatile swelling agent. The resultant silicone elastomer-solvent mixture is in the form of a gel, herein as “silicone elastomer gel”.

The silicone elastomer gel is present in the composition, according to the invention, at a concentration, by weight, from about 1% to about 20%, and in some embodiments, from about 5% to about 10%, and in some embodiments, from about 7% to about 15% by weight, based on the total weight of the composition. Thus, in various embodiments, the silicone elastomer gel comprising at least one emulsifying elastomer is present in a cosmetic composition according to the disclosure in a weight percent amount from about 1, 2, 3, 4, 5, 6, 7, 8, 9, 10, 11, 12, 13, 14, 15, 16, 17, 18, 19 to about 20 percent by weight, including increments and ranges there between.

Emulsifying silicone elastomer gels may be selected from emulsifying/self-emulsifying polyether silicone elastomers, polyglyceryl silicone elastomers, polyether dimethicone copolymers, and mixtures thereof. The emulsifying silicone elastomers may include functional groups selected from the group consisting of polyglyceryl, polyethylene glycol, or polypropylene glycol. Suitable examples of commercially available emulsifying silicone elastomer water-in-oil emulsifiers, include, but are not limited to, dimethicone (and) dimethicone/PEG-10/15 crosspolymer, dimethicone (and) dimethicone/polyglycerin-3 crosspolymer, polyether-modified crosslinked silicone polymers in dimethicone, including, for example, but not limited to dimethicone (and) PEG-15/lauryl polydimethylsiloxyethyl dimethicone crosspolymer.

The at least one emulsifying silicone elastomer is present in the compositions, according to the invention, at a concentration, from about 1% to about 20%, and in some embodiments, from about 2% to about 18% by weight, and in some embodiments, from about 7% to about 15% by weight of the cosmetic composition, and in some embodiments from about 3% to about 10%. Thus, in various embodiments, emulsifying silicone elastomer is present in a cosmetic composition according to the disclosure in a weight percent amount from about 1, 2, 3, 4, 5, 6, 7, 8, 9, 10, 11, 12, 13, 14, 15, 16, 17, 18, 19 to about 20 percent by weight, including increments and ranges there between.

Co-Emulsifier

In accordance with certain of the various embodiments, compositions according to the disclosure may comprise at least one co-emulsifier, comprising, for example, PEG-10 dimethicone, dimethicone (and) PEG/PPG-18/18 dimethicone, PEG-9 polydimethylsiloxyethyl dimethicone (and) PEG-9 available, and lauryl PEG-9 polydimethylsiloxyethyl dimethicone.

The at least one co-emulsifier, when present, is present in the compositions, according to the invention, at a concentration, from about 0.1% to about 10%, and in some embodiments, from about 0.1% to about 5% by weight, and in some embodiments, from about 0.4% to about 0.8% by weight of the cosmetic composition. Thus, in various embodiments, the at least one co-emulsifier, when present, is present in a cosmetic composition according to the disclosure in a weight percent amount from about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0 to about 10.0 percent by weight, including increments and ranges there between.

Non-Swelling, Non-Emulsifying Silicone Elastomer

In accordance with certain of the various embodiments, the cosmetic composition of the present disclosure may include at least one non-swelling non-emulsifying silicone elastomer selected from polyether-modified silicone crosslinked polymers in dimethicone. Suitable examples of commercially available non-swelling non-emulsifying silicone elastomers include, but are not limited to dimethicone (and) dimethicone/vinyl dimethicone crosspolymer, dimethicone (and) dimethicone/vinyl dimethicone crosspolymer, methyl trimethicone (and) dimethicone/vinyl dimethicone crosspolymer, diphenylsiloxy phenyl trimethicone (and) dimethicone/phenyl vinyl dimethicone crosspolymer.

The at least one non-swelling non-emulsifying silicone elastomer is present in the compositions, according to the invention, at a concentration, from about 1% to about 20%, and in some embodiments, from about 2% to about 18% by weight, and in some embodiments, from about 7% to about 15% by weight of the cosmetic composition. Thus, in various embodiments, the non-swelling non-emulsifying silicone elastomer is present in a cosmetic composition according to the disclosure in a weight percent amount from about 1, 2, 3, 4, 5, 6, 7, 8, 9, 10, 11, 12, 13, 14, 15, 16, 17, 18, 19 to about 20 percent by weight, including increments and ranges there between.

Stabilizing Spherical Powder

The cosmetic composition of the present disclosure includes at least one stabilizing spherical cosmetic powder. Representative stabilizing spherical cosmetic powders may be selected, in some embodiments, from polymer based microspheres, in particular, plastic microspheres, that include, but are not limited to, methyl methacrylate crosspolymer, HDI/trimethyl hexyllactone crosspolymer, Ethylene/Methacrylate Copolymer, polylactic acid, polymethylsilsesquioxane, polymethyl methylacrlyate, methylmethacrylate crosspolymer, ethylene, acrylic acid copyolymer, aluminimum chlorohudrate, polyethylene, acrylates/ethylhexyl acrylate crosspolymer (and) sodiumpolyacrylate, polylactic acid (and) polyglyceryl-5 laurate, and combinations thereof.

Stabilizing spherical cosmetic powders are present in the compositions, according to the invention, at a concentration, from about 1% to about 15%, and in some embodiments, from about 1% to about 12% by weight, and in some embodiments, from about 2% to about 7% by weight of the cosmetic composition. Thus, in various embodiments, the stabilizing spherical powders is present in a cosmetic composition according to the disclosure in a weight percent amount from about 1, 2, 3, 4, 5, 6, 7, 8, 9, 10, 11, 12, 13, 14, to about 15 percent by weight, including increments and ranges there between.

Aqueous Phase

The aqueous phase of the W/O emulsion cosmetic composition is at a concentration, by weight, of about 50% to about 90%, and in some embodiments, from about 55% to about 70%, and in some embodiments, from about 60% to about 65% based upon weight of the cosmetic composition. The aqueous phase present in the cosmetic composition, according to the disclosure, includes water and other water-soluble ingredients, such as, for example, one or more water soluble actives, pH-adjusting agents, glycols, lower alcohols (e.g. ethanol, propanediol), chelating agents, and preservatives. In some embodiments, the aqueous phase includes one or more non-swelling non-emulsifying silicone elastomer and at least one stabilizing agent, particularly a spherical powder. Thus, in various embodiments, the aqueous phase is present in a composition in a weight percent amount from about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 to about 90 percent by weight, including increments and ranges there between.

Water is present at a concentration, by weight, of from about 20% to about 65%, and in some embodiments, from about 55% to about 65%, and in some embodiments, from about 60% to about 65% based upon weight of the cosmetic composition. Thus, in accordance with the various embodiments, water is present in a composition according to the disclosure in a weight percent amount from about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, to about 65 percent by weight, including increments and ranges there between.

The water used may be sterile demineralized water and/or a floral water, such as rose water, cornflower water, chamomile water or lime water, and/or a natural thermal or mineral water, such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace element, such as zinc, copper, magnesium, etc., reconstituting the characteristics of a thermal water.

Aerogels

According to some embodiments herein, the compositions and methods include use of an aerogel, for example, a silica aerogel such as silica silylate.

As used here, “silica aerogels” are porous materials obtained by replacing (by drying) the liquid component of a silica gel with air. Silica aerogels are generally synthesized via a sol-gel process in a liquid medium and then dried, usually by extraction with a supercritical fluid, such as, but not limited to, supercritical carbon dioxide (CO₂). This type of drying makes it possible to avoid shrinkage of the pores and of the material. The sol-gel process and the various drying processes are described in detail in Brinker, C. J., and Scherer, G. W., Sol-Gel Science: New York: Academic Press, 1990.

Hydrophobic silica aerogel particles have a specific surface area per unit of mass (SM) ranging from about 500 to about 1500 m₂/g, or alternatively from about 600 to about 1200 m₂/g, or alternatively from about 600 to about 800 m₂/g, and a size expressed as the mean volume diameter (D[0.5]), ranging from about 1 to about 30 μm, or alternatively from about 5 to about 25 μm, or alternatively from about 5 to about 20 μm, or alternatively from about 5 to about 15 μm. The specific surface area per unit of mass may be determined via the BET (Brunauer-Emmett-Teller) nitrogen absorption method described in the Journal of the American Chemical Society, vol. 60, page 309, February 1938, corresponding to the international standard ISO 5794/1. The BET specific surface area corresponds to the total specific surface area of the particles under consideration.

Suitable examples of hydrophobic silica aerogels, may include, but are not limited to, the aerogels sold under the tradenames of VM-2260 (INCI name: Silica silylate) and VM-2270 (INCI name: Silica silylate), both available from Dow Corning Corporation (Midland, Mich.). The particles of VM-2260 have a mean size of about 1000 microns and a specific surface area per unit of mass ranging from 600 to 800 m²/g. The particles of VM-2270 have a mean size ranging from 5-15 microns and a specific surface area per unit of mass ranging from 600 to 800 m²/g. Another suitable example of a hydrophobic silica aerogel may include, but is not limited to, the aerogels commercially available from Cabot Corporation (Billerica, Mass.) under the tradename of Aerogel TLD 201, Aerogel OGD 201 and Aerogel TLD 203, Enova Aerogel MT 1100 and Enova Aerogel MT 1200.

When present, aerogels are present in the compositions, according to the invention, at a concentration, from about 0.1% to about 5%, and in some embodiments, from about 0.1% to about 2% by weight, and in some embodiments, from about 0.4% to about 0.8% by weight of the cosmetic composition. In some examples the aerogels are present in an amount that is not more than 0.6%. Thus, in various embodiments, the aerogels, when present, are present in a cosmetic composition according to the disclosure in a weight percent amount from about 0.1, 0.2, 0.3, 0.4, 0.5, 0.6, 0.7, 0.8, 0.9, 1.0, 2.0, 3.0, 4.0 to about 5.0 percent by weight, including increments and ranges there between.

Actives

Water-Soluble Active Ingredient

The compositions according to the disclosure may include one more of an active selected from but not limited to: UV protectant actives (as more fully described below), active ingredients for artificially tanning and/or browning the skin (self-tanning agents) and more particularly dihydroxyacetone (DHA), anti-aging and/or anti-oxidation agents, free-radical scavengers, keratolytic agents, vitamins (e.g., Vitamin E and derivatives thereof), anti-elastase and anti-collagenase agents, peptides, fatty acid derivatives, steroids, trace elements, extracts of algae and of planktons, enzymes and coenzymes, flavonoids and ceramides, hydroxy acids and mixtures thereof, and enhancing agents. These ingredients may be soluble or dispersible in any one or more of suitable phases of the composition (i.e., aqueous and/or fatty (oil) phase.)

At least the aqueous phase present in the composition, according to the disclosure, includes at least one water-soluble active ingredient. The water-soluble active ingredients can be present in their synthetic chemical compound forms, or alternatively as integral part of botanical extracts. Suitable examples of water-soluble ingredients, include, but are not limited to, (1) phenolic and polyphenolic compounds, and (2) other non-phenolic compounds.

The active will be present in the composition according to the invention, at a concentration, from about 0.01% to 80%, in some embodiments from about 0.1% to 60%, and in some embodiments from about 0.5% to 50% by weight, all weights based on the total weight of the composition. Thus, in various embodiments, the active is present in a composition in a weight percent amount from 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 40.0, 50.0, 60.0, 70.0, and 80.0 percent by weight, including increments there between.

UV Protectant Actives [INVENTORS: please supplement or revise this section on UV actives as may be appropriate]

Inorganic UV Filters

The composition, according to the present invention, may comprise at least one inorganic UV filter. If two or more inorganic UV filters are used, they may be the same or different.

The inorganic UV filter used for the present invention may be active in the UV-A and/or UV-B region. The inorganic UV filter may be hydrophilic and/or lipophilic. The inorganic UV filter is in some embodiments insoluble in solvents, such as water, and ethanol commonly used in cosmetics.

It is in some embodiments desirable that the inorganic UV filter be in the form of a fine particle such that the mean (primary) particle diameter thereof ranges from 1 nm to 50 nm, and in some embodiments 5 nm to 40 nm, and in some embodiments 10 nm to 30 nm. The mean (primary) particle size or mean (primary) particle diameter here is an arithmetic mean diameter.

The inorganic UV filter can be selected from the group consisting of silicone carbide, metal oxides which may or may not be coated, and mixtures thereof. And in some embodiments, the inorganic UV filters are selected from pigments (mean size of the primary particles: generally from 5 nm to 50 nm, and in some embodiments from 10 nm to 50 nm) formed of metal oxides, such as, for example, pigments formed of titanium oxide (amorphous or crystalline in the rutile and/or anatase form), iron oxide, zinc oxide, zirconium oxide, or cerium oxide, which are all UV photoprotective agents that are well known per se. And in some embodiments, the inorganic UV filters are selected from titanium oxide, zinc oxide, and in some embodiments, titanium oxide.

The inorganic UV filter may or may not be coated. The inorganic UV filter may have at least one coating. The coating may comprise at least one compound selected from the group consisting of alumina, silica, aluminum hydroxide, silicones, silanes, fatty acids or salts thereof (such as sodium, potassium, zinc, iron, or aluminum salts), fatty alcohols, lecithin, amino acids, polysaccharides, proteins, alkanolamines, waxes, such as beeswax, (meth)acrylic polymers, organic UV filters, and (per)fluoro compounds. It is in some embodiments desirable for the coating to include at least one organic UV filter. As the organic UV filter in the coating, a dibenzoylmethane derivative, such as butyl methoxydibenzoylmethane (Avobenzone) and 2,2′-Methylenebis[6-(2H-Benzotriazol-2-yl)-4-(1,1,3,3-Tetramethyl-Butyl) Phenol] (Methylene Bis-Benzotriazolyl Tetramethylbutylphenol) marketed as “TINOSORB M” by BASF, may be desirable.

Of course, the inorganic UV filters made of metal oxides may, before their treatment with silicones, have been treated with other surfacing agents, in particular with cerium oxide, alumina, silica, aluminum compounds, silicone compounds, or their mixtures. The coated inorganic UV filter may have been prepared by subjecting the inorganic UV filter to one or more surface treatments of a chemical, electronic, mechano-chemical, and/or mechanical nature with any of the compounds as described above, as well as polyethylenes, metal alkoxides (titanium or aluminum alkoxides), metal oxides, sodium hexametaphosphate, and those shown, for example, in Cosmetics & Toiletries, February 1990, Vol. 105, pp. 53-64.

The coated inorganic UV filters may be titanium oxides coated: with silica, such as the product “Sun veil” from Ikeda, and “Sunsil TIN 50” from Sunjin Chemical; with silica and with iron oxide, such as the product “Sunveil F” from Ikeda; with silica and with alumina, such as the products “Microtitanium Dioxide MT 500 SA” from Tayca, “Tioveil” from Tioxide, and “Mirasun TiW 60” from Rhodia; with alumina, such as the products “Tipaque TTO-55 (B)” and “Tipaque TTO-55 (A)” from Ishihara, and “UVT 14/4” from Kemira; with alumina and with aluminum stearate, such as the product “Microtitanium Dioxide MT 100 T, MT 100 TX, MT 100 Z or MT-01” from Tayca, the products “Solaveil CT-10 W” and “Solaveil CT 100” from Uniqema, and the product “Eusolex T-AVO” from Merck; with alumina and with aluminum laurate, such as the product “Microtitanium Dioxide MT 100 S” from Tayca; with iron oxide and with iron stearate, such as the product “Microtitanium Dioxide MT 100 F” from Tayca; with zinc oxide and with zinc stearate, such as the product “BR351” from Tayca; with silica and with alumina and treated with a silicone, such as the products “Microtitanium Dioxide MT 600 SAS”, “Microtitanium Dioxide MT 500 SAS”, and “Microtitanium Dioxide MT 100 SAS” from Tayca; with silica, with alumina and with aluminum stearate and treated with a silicone, such as the product “STT-30-DS” from Titan Kogyo; with silica and treated with a silicone, such as the product “UV-Titan X 195” from Kemira; with alumina and treated with a silicone, such as the products “Tipaque TTO-55 (S)” from Ishihara or “UV Titan M 262” from Kemira; with triethanolamine, such as the product “STT-65-S” from Titan Kogyo; with stearic acid, such as the product “Tipaque TTO-55 (C)” from Ishihara; or with sodium hexametaphosphate, such as the product “Microtitanium Dioxide MT 150 W” from Tayca. Other titanium oxide pigments treated with a silicone are, and in some embodiments TiO₂ treated with octyltrimethylsilane and for which the mean size of the individual particles is from 25 and 40 nm, such as that marketed under the trademark “T 805” by Degussa Silices, TiO₂ treated with a polydimethylsiloxane and for which the mean size of the individual particles is 21 nm, such as that marketed under the trademark “70250 Cardre UF TiO₂Si₃” by Cardre, and anatase/rutile TiO₂ treated with a polydimethylhydrosiloxane and for which the mean size of the individual particles is 25 nm, such as that marketed under the trademark “Microtitanium Dioxide USP Grade Hydrophobic” by Color Techniques.

And in some embodiments, the following coated TiO₂ can be used as the coated inorganic UV filter: Stearic acid (and) Aluminum Hydroxide (and) TiO₂, such as the product “MT-100 TV” from Tayca, with a mean primary particle diameter of 15 nm; Dimethicone (and) Stearic Acid (and) Aluminum Hydroxide (and) TiO₂, such as the product “S A-TTO-S4” from Miyoshi Kasei, with a mean primary particle diameter of 15 nm; Silica (and) TiO₂, such as the product “MT-100 WP” from Tayca, with a mean primary particle diameter of 15 nm; Dimethicone (and) Silica (and) Aluminum Hydroxide (and) TiO₂, such as the product “MT-Y02” and “MT-Y-110 M3S” from Tayca, with a mean primary particle diameter of 10 nm; Dimethicone (and) Aluminum Hydroxide (and) TiO₂, such as the product “SA-TTO-S3” from Miyoshi Kasei, with a mean primary particle diameter of 15 nm; Dimethicone (and) Alumina (and) TiO₂, such as the product “UV TITAN MI 70” from Sachtleben, with a mean primary particle diameter of 15 nm; and Silica (and) Aluminum Hydroxide (and) Alginic Acid (and) TiO₂, such as the product “MT-100 AQ” from Tayca, with a mean primary particle diameter of 15 nm. In terms of UV filtering ability, TiO₂ coated with at least one organic UV filter is more desirable. For example, Avobenzone (and) Stearic Acid (and) Aluminum Hydroxide (and) TiO₂, such as the product “HXMT-100ZA” from Tayca, with a mean primary particle diameter of 15 nm, can be used.

The uncoated titanium oxide pigments are, for example, marketed by Tayca under the trademarks “Microtitanium Dioxide MT500B” or “Microtitanium Dioxide MT600B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”. The uncoated zinc oxide pigments are, for example: those marketed under the trademark “Z-cote” by Sunsmart; those marketed under the trademark “Nanox” by Elementis; and those marketed under the trademark “Nanogard WCD 2025” by Nanophase Technologies. The coated zinc oxide pigments are, for example: those marketed under the trademark “Oxide Zinc CS-5” by Toshiba (ZnO coated with polymethylhydrosiloxane); those marketed under the trademark “Nanogard Zinc Oxide FN” by Nanophase Technologies (as a 40% dispersion in Finsolv TN, C₁₂-C₁₅ alkyl benzoate); those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane); those marketed under the trademark “NFD Ultrafine ZnO” by Daikin (ZnO coated with phosphate of perfiuoroalkyl and a copolymer based on perfluoroalkylethyl as a dispersion in cyclopentasiloxane); those marketed under the trademark “SPD-Z1” by Shin-Etsu (ZnO coated with a silicone-grafted acrylic polymer dispersed in cyclodimethylsiloxane); those marketed under the trademark “Escalol Z100” by ISP (alumina-treated ZnO dispersed in an ethylhexyl methoxycinnamate/PVP-hexadecene copolymer/methicone mixture); those marketed under the trademark “Fuji ZnO-SMS-10” by Fuji Pigment (ZnO coated with silica and polymethylsilsesquioxane); and those marketed under the trademark “Nanox Gel TN” by Elementis (ZnO dispersed at 55% in C₁₂-C₁₅ alkyl benzoate with hydroxystearic acid polycondensate). The uncoated cerium oxide pigments are marketed, for example, under the trademark “Colloidal Cerium Oxide” by Rhone-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ”, and “Nanogard WCD 2006 (FE 45R)”, or by Mitsubishi under the trademark “TY-220”. The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345”, and “Nanogard FE 45 BL”, or by BASF under the trademark “Oxyde de fer transparent”.

Mention may also be made of mixtures of metal oxides, in particular, of titanium dioxide and of cerium dioxide, including a mixture of equal weights of titanium dioxide coated with silica and of cerium dioxide coated with silica marketed by Ikeda under the trademark “Sunveil A”, and also a mixture of titanium dioxide and of zinc dioxide coated with alumina, with silica and with silicone, such as the product “M 261” marketed by Kemira, or coated with alumina, with silica and with glycerol, such as the product “M 211” marketed by Kemira. Coated inorganic UV filters are desirable, because the UV filtering effects of the inorganic UV filters can be enhanced. In addition, the coating(s) may help uniformly or homogeneously disperse the UV filters in the composition, according to the present invention.

Organic UV Filters

The compositions according to the disclosure may comprise at least one organic UV filter. If two or more organic UV filters are used, they may be the same or different. The organic UV filter used for the present invention may be active in the UV-A and/or UV-B region. The organic UV filter may be hydrophilic and/or lipophilic. The organic UV filter may be solid or liquid. The terms “solid” and “liquid” mean solid and liquid, respectively, at 25° C. under 1 atm.

The organic UV filter can be selected from the group consisting of anthranilic compounds; dibenzoylmethane compounds; cinnamic compounds; salicylic compounds; camphor compounds; benzophenone compounds; β,β-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures thereof.

Mention may be made, as examples of the organic UV filter(s), of those denoted below under their INCI names, and mixtures thereof. Anthranilic compounds: Menthyl anthranilate, marketed under the trademark “Neo Heliopan MA” by Haarmann and Reimer. Dibenzoylmethane compounds: Butyl methoxydibenzoylmethane, marketed in particular under the trademark “Parsol 1789” by Hoffmann-La Roche; and isopropyl dibenzoylmethane. Cinnamic compounds: Ethylhexyl methoxycinnamate, marketed in particular under the trademark “Parsol MCX” by Hoffmann-La Roche; isopropyl methoxycinnamate; isopropoxy methoxycinnamate; isoamyl methoxycinnamate, marketed under the trademark “Neo Heliopan E 1000” by Haarmann and Reimer; cinoxate (2-ethoxyethyl-4-methoxy cinnamate); DEA methoxycinnamate; diisopropyl methylcinnamate; and glyceryl ethylhexanoate dimethoxycinnamate. Salicylic compounds: Homosalate (homomentyl salicylate), marketed under the trademark “Eusolex HMS” by Rona/EM Industries; ethylhexyl salicylate, marketed under the trademark “Neo Heliopan OS” by Haarmann and Reimer; glycol salicylate; butyloctyl salicylate; phenyl salicylate; dipropyleneglycol salicylate, marketed under the trademark “Dipsal” by Scher; and TEA salicylate, marketed under the trademark “Neo Heliopan TS” by Haarmann and Reimer. Camphor compounds, in particular, benzylidenecamphor derivatives: 3-benzylidene camphor, manufactured under the trademark “Mexoryl SD” by Chimex; 4-methylbenzylidene camphor, marketed under the trademark “Eusolex 6300” by Merck; benzylidene camphor sulfonic acid, manufactured under the trademark “Mexoryl SL” by Chimex; camphor benzalkonium methosulfate, manufactured under the trademark “Mexoryl SO” by Chimex; terephthalylidene dicamphor sulfonic acid, manufactured under the trademark “Mexoryl SX” by Chimex; and polyacrylamidomethyl benzylidene camphor, manufactured under the trademark “Mexoryl SW” by Chimex. Benzophenone compounds: Benzophenone-1 (2,4-dihydroxybenzophenone), marketed under the trademark “Uvinul 400” by BASF; benzophenone-2 (Tetrahydroxybenzophenone), marketed under the trademark “Uvinul D50” by BASF; Benzophenone-3 (2-hydroxy-4-methoxybenzophenone) or oxybenzone, marketed under the trademark “Uvinul M40” by BASF; benzophenone-4 (hydroxymethoxy benzophonene sulfonic acid), marketed under the trademark “Uvinul MS40” by BASF; benzophenone-5 (Sodium hydroxymethoxy benzophenone Sulfonate); benzophenone-6 (dihydroxy dimethoxy benzophenone); marketed under the trademark “Helisorb 11” by Norquay; benzophenone-8, marketed under the trademark “Spectra-Sorb UV-24” by American Cyanamid; benzophenone-9 (Disodium dihydroxy dimethoxy benzophenonedisulfonate), marketed under the trademark “Uvinul DS-49” by BASF; and benzophenone-12, and n-hexyl 2-(4-diethylamino-2-hydroxybenzoyl)benzoate (UVINUL A+ by BASF). β,β-Diphenylacrylate compounds: Octocrylene, marketed in particular under the trademark “Uvinul N539” by BASF; and Etocrylene, marketed in particular under the trademark “Uvinul N35” by BASF. Triazine compounds: Diethylhexyl butamido triazone, marketed under the trademark “Uvasorb HEB” by Sigma 3V; 2,4,6-tris(dineopentyl 4′-aminobenzalmalonate)-s-triazine, bis-ethylhexyloxyphenol methoxyphenyl triazine marketed under the trademark <<TINOSORB S>> by CIBA GEIGY, and ethylhexyl triazone marketed under the trademark <<UVTNUL T150>> by BASF. Benzotriazole compounds, in particular, phenylbenzotriazole derivatives: 2-(2H-benzotriazole-2-yl)-6-dodecyl-4-methylpheno, branched and linear; and those described in U.S. Pat. No. 5,240,975. Benzalmalonate compounds: Dineopentyl 4′-methoxybenzalmalonate, and polyorganosiloxane comprising benzalmalonate functional groups, such as polysilicone-15, marketed under the trademark “Parsol SLX” by Hoffmann-LaRoche. Benzimidazole compounds, in particular, phenylbenzimidazole derivatives: Phenylbenzimidazole sulfonic acid, marketed in particular under the trademark “Eusolex 232” by Merck, and disodium phenyl dibenzimidazole tetrasulfonate, marketed under the trademark “Neo Heliopan AP” by Haarmann and Reimer. Imidazoline compounds: Ethylhexyl dimethoxybenzylidene dioxoimidazoline propionate. Bis-benzoazolyl compounds: The derivatives as described in EP-669,323 and U.S. Pat. No. 2,463,264. Para-aminobenzoic acid compounds: PABA (p-aminobenzoic acid), ethyl PABA, Ethyl dihydroxypropyl PABA, pentyl dimethyl PABA, ethylhexyl dimethyl PABA, marketed in particular under the trademark “Escalol 507” by ISP, glyceryl PABA, and PEG-25 PABA, marketed under the trademark “Uvinul P25” by BASF. Methylene bis-(hydroxyphenylbenzotriazol) compounds, such as 2,2′-methylenebis[6-(2H-benzotriazol-2-yl)-4-methyl-phenol] marketed in the solid form under the trademark “Mixxim BB/200” by Fairmount Chemical, 2,2′-methylenebis[6-(2H-benzotriazol-2-yl)-4-(I,I,3,3-tetramethylbutyl)phenol] marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184, and EP-893,119, and Drometrizole trisiloxane, marketed under the trademark “Silatrizole” by Rhodia Chimie or- “Mexoryl XL” by L'Oreal. Benzoxazole compounds: 2,4-bis[5-I(dimethylpropyl)benzoxazol-2-yl-(4-phenyl)imino]-6-(2-ethylhexyl)imino-I,3,5-triazine, marketed under the trademark of Uvasorb K2A by Sigma 3V. Screening polymers and screening silicones: The silicones described in WO 93/04665. Dimers derived from a-alkylstyrene: The dimers described in DE-19855649. 4,4-Diarylbutadiene compounds: I,I-dicarboxy(2,2′-dimethylpropyl)-4,4-diphenylbutadiene.

It is in some embodiments desirable that the organic UV filter(s) be selected from the group consisting of: butyl methoxydibenzoylmethane, ethylhexyl methoxycinnamate, homosalate, ethylhexyl salicylate, octocrylene, phenylbenzimidazole sulfonic acid, benzophenone-3, benzophenone-4, benzophenone-5, n-hexyl 2-(4-diethylamino-2-hydroxybenzoyl)benzoate, I, r-(I,4-piperazinediyl)bis[I-[2-[4-(diethylamino)-2-hydroxybenzoyl]phenyl]-methanone 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone, 2,4,6-tris(dineopentyl 4′-aminobenzalmalonate)-s-triazine, 2,4,6-tris(diisobutyl 4′-aminobenzalmalonate)-s-triazine, 2,4-bis-(n-butyl 4′-aminobenzalmalonate)-6-[(3-{1,3,3,3-tetramethyl-1-[(trimethylsilyloxy]-disiloxanyl}propyl)amino]-s-triazine, 2,4,6-tris-(di-phenyl)-triazine, 2,4,6-tris-(ter-phenyl)-triazine, methylene bis-benzotriazolyl tetramethylbutylphenol, drometrizole trisiloxane, polysilicone-15, dineopentyl 4′-methoxybenzalmalonate, I,I-dicarboxy(2,2′-dimethylpropyl)-4,4-diphenylbutadiene, 2,4-bis[5-I (dimethylpropyl)benzoxazol-2-yl-(4-phenyl)imino]-6-(2-ethylhexyl)imino-I,3,5-triazine, camphor benzylkonium methosulfate, and mixtures thereof.

Other Ingredients

Emollient

The compositions according to the disclosure may include one more of an emollient, including polar and/or non-polar emollients. Suitable polar emollients include, but are not limited to, polyol esters and polyol ethers such as linear or branched chained polyglycerol esters and polyglycerol ethers. Particularly suitable emollients for use as the first emollient include caprylic/capric triglyceride, isopropyl myristate, PPG-3 myristyl ether, isopropyl palmitate, dibutyl adipate, propyleneglycol dicaprylate/dicaprate, cocoglyceride, cetearyl isononanoate, isopropyl myristate, isodecyl neopentanoate, tridecyl neopentanoate, C₁₂₋₁₅ alkyl benzoate, isopropyl lauroyl sarsosinate, phenethyl benzoate and mixtures thereof. Other suitable emollients include any oil soluble emollients having high or medium polarity moieties.

Emollients may be present in the composition according to the invention, at a concentration, from about 1% to 25%, in some embodiments from about 2% to 18%, and in some embodiments from about 5% to 10% by weight, all weights based on the total weight of the composition. Thus, in various embodiments, an emollient may be present in a composition in a weight percent amount from 1, 2, 3, 4, 5, 6, 7, 8, 9, 10, 11, 12, 13, 14, 15, 16, 17, 18, 19, 20,21, 22, 23, 24 to 25 percent by weight, including increments and ranges there between.

Hydrating Agent

The compositions according to the disclosure may include one more of a hydrating agent.

Suitable examples of the hydrating agent, include polyols, for example, glycerol, glycols, such as butylene glycol, propylene glycol, isoprene glycol, dipropylene glycol, hexylene glycol and polyethylene glycols, sorbitol, sugars, such as glucose, and mixtures thereof. According to one desirable embodiment of the invention, the polyol chosen is glycerol, dipropylene glycol or mixtures thereof, or a mixture of glycerol and/or of dipropylene glycol and of one or more other polyols especially chosen from those indicated above: butylene glycol, propylene glycol, isoprene glycol, hexylene glycol, polyethylene glycols, sorbitol, sugars, methylpropanediol and 1,3-propanediol and mixtures thereof. A particularly suitable polyol for use with the present invention is glycerin.

Hydrating agents may be present in the composition according to the invention, at a concentration, from about 1% to 25%, in some embodiments from about 2% to 18%, and in some embodiments from about 5% to 10% by weight, all weights based on the total weight of the composition. Thus, in various embodiments, an hydrating agent may be present in a composition in a weight percent amount from 1, 2, 3, 4, 5, 6, 7, 8, 9, 10, 11, 12, 13, 14, 15, 16, 17, 18, 19, 20,21, 22, 23, 24 to 25 percent by weight, including increments and ranges there between.

Preservative System

The compositions according to the disclosure may include one or more preservatives or a preservative system. In a desirable embodiment, the preservative system may include organic acids, parabens, formaldehyde donors, phenol derivatives, quaternary ammoniums, alcohols, isothiazolones, and combinations thereof.

Examples of organic acid preservative systems include, but are not limited to, sodium benzoate, potassium sorbate, benzoic acid and dehydroaceticic acid, sorbic acid, and combinations thereof. A desirable organic acid preservative system may include a mixture of sodium benzoate and potassium sorbate. Examples of paraben preservative systems include, but are not limited to, alkyl para-hydroxybenzoates, wherein the alkyl radical has from 1, 2, 3, 4, 5 or 6 carbon atoms and, in some embodiments, from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Examples of formaldehyde donor preservative systems include, but are not limited to, 1,3-Dimethylol-5,5-dimethylhydantoin (DMDM hydantoin), imidazolidinyl urea, gluteraldehyde, and combinations thereof. Examples of quaternary ammonium preservative systems include, but are not limited to, benzalkonium chloride, methene ammonium chloride, benzethonium chloride, and combinations thereof. Examples of alcohol preservative systems include, but are not limited to, ethanol, benzyl alcohol, dichlorobenzyl alcohol, phenoxyethanol, and combinations thereof. Examples of isothiazolone preservative systems include, but are not limited to, methylchloroisothiazolinone, methylisothiazolinone, and combinations thereof.

Other suitable preservatives for preservative systems include, but are not limited to, chloracetamide, triclosan and iodopropynyl butylcarbamate, pyridine derivatives (e.g., pyrithione and zinc pyrithione), chlorphenesin, phenyl mercuric salts, phenoxyethanol, and other known preservative systems.

Preservatives may be present in the composition according to the invention, at a concentration, from about 0.01% to 25%, in some embodiments from about 0.1% to 20%, and in some embodiments from about 0.5% to 15% by weight, all weights based on the total weight of the composition. Thus, in various embodiments, a preservative may be present in a composition in a weight percent amount from 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0 to 25.0 percent by weight, including increments and ranges there between.

Fillers

The compositions according to the disclosure may include one more of a filler. The fillers may be of mineral or organic origin, natural or synthetic in nature in order to provide oil absorption or optical effects. Oil absorption fillers may impart a matte effect and non-greasy feeling onto the skin. Optical effects fillers may impart a soft-focus/haze/blur effect to the skin, provide the skin with a more uniform appearance, reduce the appearance of skin imperfections or discoloration, or reduce the visibility of pores.

Mention may be made as examples of oil-absorbing fillers: mica, zea may (corn) starch, magnesium oxide, nylon-12, nylon-66, cellulose, polyethylene, talc, talc (and) methicone, talc (and) dimethicone, perlite, sodium silicate, pumice, ptfe, polymethyl methacrylate, oryza sativa (rice) starch, aluminum starch octenylsuccinate, potato starch modified, alumina, calcium sodium borosilicate, magnesium carbonate, hydrated silica, dimethicone/vinyl dimethicone crosspolymer, sodium carboxylmethyl starch. According to one preferred embodiment, the oil-absorbing filler comprises spherical microparticles of porous silica having a mean particle size from 0.5 to 20 μm whose INCI name is silica sold by the company JCG Catalysts and Chemicals under the name Spheron L-1500. According to another preferred embodiment, the oil absorbing filler comprises hydrophobic aerogel particles whose INCI name is silica silylate sold by Dow Corning under the name VM-2270 Aerogel Fine Particles.

Mention may be made as examples of optical effects fillers: bismuth oxychloride, silica silylate, boron nitride, iron oxide, calcium carbonate, calcium sulfate (and) iron oxides, sodium potassium aluminum silicate.

Mention may be made as examples of fillers which provide both oil-absorbing and optical effects: silica, silica (and) methicone, silica (and) dimethicone, polysilicone-22, polysilicone-8, polysilicone-11, methyl metacrylate crosspolymer, polymethylsilsesquioxane, methylsilanol/silicate crosspolymer, vinyl dimethicone/methicone silsesquioxane crosspolymer, diphenyl dimethicone/vinyl diphenyl dimethicone silsesquioxane crosspolymer, and styrene/acrylates copolymer.

The filler may be present in the composition according to the invention, at a concentration, from about 0.01% to 25%, in some embodiments from about 0.1% to 20%, and in some embodiments from about 0.5% to 15% by weight, all weights based on the total weight of the composition. Thus, in various embodiments, a filler may be present in a composition in a weight percent amount from 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0 to 25.0 percent by weight, including increments and ranges there between.

SPF Boosters

The compositions according to the disclosure may comprise one or more of an SPF booster. The term “SPF booster” means a compound or composition that, when used in a composition in conjunction with a UV screening agent, increases the SPF value of the composition without increasing the amount of the UV screening agent in the composition. It is, in some embodiments, desirable that the SPF booster be made from at least one material selected from the group consisting of (co)polymers of (meth)acrylic acid, (meth)acrylates, and/or styrene; glass; silica; and mixtures thereof. As the (co)polymers of (meth)acrylic acid, (meth)acrylates, and/or styrene, mention may be made of poly(meth)acrylates, such as PMMA, a copolymer of (meth)acrylic acid and (meth)acrylates, and a copolymer of (meth)acrylic acid, (meth)acrylates, and styrene.

SPF boosters may be present in the composition according to the invention, at a concentration, from about 0.01% to 25%, in some embodiments from about 0.1% to 13%, and in some embodiments from about 0.5% to 10% by weight, all weights based on the total weight of the composition. Thus, in various embodiments, an SPF booster may be present in a composition in a weight percent amount from 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0 to 25.0 percent by weight, including increments there between.

Other Optional Ingredients

The compositions according to the disclosure may include one more of a variety of optional ingredient, selected from but not limited to, one or more standard cosmetic adjuvants chosen from: oils, waxes, organic solvents, fillers, ionic or nonionic, hydrophilic or lipophilic thickeners, softeners, humectants, opacifiers, stabilizers, emollients, silicones, antifoams, fragrances, preserving agents, surfactants, active agents, coloring agents, cationic polymers, propellants, neutralizing or pH-adjusting agents (e.g., citric acid, triethylamine (TEA) and sodium hydroxide), conditioning or softening agents (e.g., panthenol and allantoinin), extracts, such as botanical extracts, free-radical scavengers, keratolytic agents, vitamins (e.g., Vitamin E and derivatives thereof), anti-elastase and anti-collagenase agents, peptides, fatty acid derivatives, steroids, trace elements, extracts of algae and of planktons, enzymes and coenzymes, flavonoids and ceram ides, hydroxy acids and mixtures thereof, and enhancing agents. These ingredients may be soluble or dispersible in whatever phase or phases is/are present in the cosmetic composition (i.e., aqueous and/or fatty (oil) phase) or any other ingredient usually used in cosmetics and/or dermatology.

Optional Ingredients may be present in the compositions in amounts generally ranging from about 0.01% to 25%, in some embodiments from about 0.1% to 20%, and in some embodiments from about 0.5% to 15% by weight, all weights based on the total weight of the composition. Thus, in various embodiments, an SPF booster may be present in a composition in a weight percent amount from 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0 to 25.0 percent by weight, including increments there between.

Those skilled in the art will take care to select the optional additional compound(s) mentioned above and/or the amounts thereof such that the advantageous properties intrinsically associated with the compositions, in accordance with the invention, are not, or are not substantially, adversely affected by the envisaged addition(s). Those skilled in the art will choose said active agent(s) according to the desired effect on the skin, hair, eyelashes, eyebrows, or nails.

Stable Emulsion

Compositions, according to the present disclosure, include a stable composition. By “stable” it is meant that the compositions initially have a good aesthetic appearance, including lack of a grainy texture and no visually perceptible separation of phases. In addition, the “stable” cosmetic composition, according to the present disclosure, is stable after exposure to a plurality of freeze/thaw cycles. For example, a cosmetic composition is considered stable if its viscosity is within 10-UD from the initial viscosity after 10 days of freeze/thaw cycles (−20° C. to 25° C.) and does not exhibit signs of phase separation, does not show a grainy texture and/or become inhomogeneous. [INVENTORS: do you have viscosity data to provide?]

Method [INVENTORS: please supplement or revise this methods section as may be appropriate]

The cosmetic composition is prepared by combining the oil phase ingredients (shown as phase (A) in the examples), including organic UV filters, in a vessel and heating this oil phase while gently mixing until all solids dissolved, giving a homogeneous phase. The oil phase component is observed to verify that the components are dispersed and the mixing is continued while heating until fully homogeneous.

Once the oil phase components are mixed and homogenous, the water or aqueous phase ingredients (shown as phase (B) in the examples) are combined in a separate vessel and heated to, for example, 50-60° C. The water phase ingredients are mixed until the solids dissolved upon observation. In one embodiment, water-soluble UV filters can be added in the water phase.

Once the water phase is mixed and the components are dissolved, the water phase is added to the oil phase over a period of time, for example, for a period of 5-10 min. while mixing. The components are vigorously mixed, for example, with a propeller and/or rotor-stator, until a smooth and homogeneous emulsion is obtained.

In certain embodiments, an additional phase and/or other temperature-sensitive ingredients can be added to the blended oil and water phases at lower temperatures and mixed well until smooth and homogeneous.

The following examples are intended to further illustrate the present invention. They are not intended to limit the invention in any way. Unless otherwise indicated, all parts are by weight.

### EXAMPLES

The inventive formulations were made generally in accordance with the method described above. As further described herein below, the inventive formulations were evaluated by addition of one or more supplemental components selected from stabilizing spherical powders and aerogels to evaluate haze/transmission aesthetic properties and to determine freeze/thaw stability.

TABLE 1
Inventive Non-Sunscreen Formulation

| | | | Inventive | Inventive |
|---|---|---|---|---|
| | | | Ex 1 | Ex 2 |
| Rank | Phase | INCI US Name | % | % |
| 1 | A | DIMETHICONE | 5 | 5 |
| 2 | A | PEG-9 | 0.9 | 0.9 |
| | | POLYDIMETHYLSILOXY- | | |
| | | ETHYL DIMETHICONE | | |
| 3 | A | DIMETHICONE (and) | 5 | 5 |
| | | DIMETHICONE/PEG-10/15 | | |
| | | CROSSPOLYMER | | |
| 4 | A | PENTAERYTHRITYL | 3 | 3 |
| | | TETRAETHYLHEXANOATE | | |
| 5 | B | WATER (QS) | 49.55 | 49.55 |
| 6 | B | GLYCERIN | 7 | 7 |
| 7 | B | PROPANEDIOL | 3 | 3 |
| 8 | B | CAPRYLYL GLYCOL | 0.3 | 0.3 |
| 9 | B | PHENOXYETHANOL | 0.5 | 0.5 |
| 10 | B | CHLORPHENESIN | 0.3 | 0.3 |
| 11 | B | DISODIUM EDTA | 0.1 | 0.1 |
| 12 | B | SODIUM CITRATE | 0.2 | 0.2 |
| 13 | B | SODIUM CHLORIDE | 0.5 | 0.5 |
| 14 | B | CITRIC ACID | 0.05 | 0.05 |
| 15 | C | DIMETHICONE | 3 | 3 |
| 16 | C | DIMETHICONE (and) | 15 | 15 |
| | | DIMETHICONE/VINYL | | |
| | | DIMETHICONE | | |
| | | CROSSPOLYMER | | |
| 17 | C | SILICA SILYLATE | 0.6 | 0.6 |
| 18 | D | POLYMETHYL- | 2 | 2 |
| | | SILSESQUIOXANE | | |
| | | Boron Nitride | | |
| | | Methyl Methacrylate Crosspolymer | | |
| | | (and) Mineral Oil | | |
| | | Nylon-12 Fluorescent Brightener | | |
| | | 230 Salt (and) Polyvinylalcohol | | |
| | | Crosspolymer | | |
| | | Nylon-12 | | |
| | | Diakalyte | | |
| | | Lauroyl Lysine | | |
| | | MICA (and) Titanium Dioxide | | |
| | | Methyl Methacrylate Crosspolymer | 4 | |
| | | HDI/Trimethylol Hexyllactone | | 4 |
| | | Crosspolymer | | |
| | | Freeze/Thaw (−20 C./25 C., | Pass | Pass |
| | | 10 cycles) - Stability | | |
| | | Particle Size (μm) | 5-10 | 10-15 |
| | | Haze (%) | 91.2 | 76.47 |
| | | Transmission (%) | 89.93 | 90.47 |

TABLE 2
Comparative Non-Sunscreen composition

| | | | Compar- | Compar- |
|---|---|---|---|---|
| | | | ative | ative |
| | | | Ex 1 | Ex 2 |
| Rank | Phase | INCI US Name | % | % |
| 1 | A | DIMETHICONE | 5 | 5 |
| 2 | A | PEG-9 | 0.9 | 0.9 |
| | | POLYDIMETHYLSILOXY- | | |
| | | ETHYL DIMETHICONE | | |
| 3 | A | DIMETHICONE (and) | 5 | 5 |
| | | DIMETHICONE/PEG-10/15 | | |
| | | CROSSPOLYMER | | |
| 4 | A | PENTAERYTHRITYL | 3 | 3 |
| | | TETRAETHYLHEXANOATE | | |
| 5 | B | WATER (QS) | 53.55 | 49.55 |
| 6 | B | GLYCERIN | 7 | 7 |
| 7 | B | PROPANEDIOL | 3 | 3 |
| 8 | B | CAPRYLYL GLYCOL | 0.3 | 0.3 |
| 9 | B | PHENOXYETHANOL | 0.5 | 0.5 |
| 10 | B | CHLORPHENESIN | 0.3 | 0.3 |
| 11 | B | DISODIUM EDTA | 0.1 | 0.1 |
| 12 | B | SODIUM CITRATE | 0.2 | 0.2 |
| 13 | B | SODIUM CHLORIDE | 0.5 | 0.5 |
| 14 | B | CITRIC ACID | 0.05 | 0.05 |
| 15 | C | DIMETHICONE | 3 | 3 |
| 16 | C | DIMETHICONE (and) | 15 | 15 |
| | | DIMETHICONE/VINYL | | |
| | | DIMETHICONE | | |
| | | CROSSPOLYMER | | |
| 17 | C | SILICA SILYLATE | 0.6 | 0.6 |
| 18 | D | POLYMETHYL- | 2 | 2 |
| | | SILSESQUIOXANE | | |
| | | Boron Nitride | | 4 |
| | | Methyl Methacrylate Crosspolymer | | |
| | | (and) Mineral Oil | | |
| | | Nylon-12 Fluorescent Brightener | | |
| | | 230 Salt (and) Polyvinylalcohol | | |
| | | Crosspolymer | | |
| | | Nylon-12 | | |
| | | Diakalyte | | |
| | | Lauroyl Lysine | | |
| | | MICA (and) Titanium Dioxide | | |
| | | Methyl Methacrylate Crosspolymer | | |
| | | HDI/Trimethylol Hexyllactone | | |
| | | Crosspolymer | | |
| | | Freeze/Thaw (−20 C./25 C., | Fail | Fail |
| | | 10 cycles) - Stability | | |
| | | Particle Size (μm) | 5-15 | 5-15 |
| | | Haze (%) | 51.73 | 80.8 |
| | | Transmission (%) | 89.7 | 86.5 |

TABLE 3
Comparative Non-Sunscreen composition

| | | | Compar- | Compar- |
|---|---|---|---|---|
| | | | ative | ative |
| | | | Ex 3 | Ex 4 |
| Rank | Phase | INCI US Name | % | % |
| 1 | A | DIMETHICONE | 5 | 5 |
| 2 | A | PEG-9 | 0.9 | 0.9 |
| | | POLYDIMETHYLSILOXY- | | |
| | | ETHYL DIMETHICONE | | |
| 3 | A | DIMETHICONE (and) | 5 | 5 |
| | | DIMETHICONE/PEG-10/15 | | |
| | | CROSSPOLYMER | | |
| 4 | A | PENTAERYTHRITYL | 3 | 3 |
| | | TETRAETHYLHEXANOATE | | |
| 5 | B | WATER (QS) | 49.55 | 49.55 |
| 6 | B | GLYCERIN | 7 | 7 |
| 7 | B | PROPANEDIOL | 3 | 3 |
| 8 | B | CAPRYLYL GLYCOL | 0.3 | 0.3 |
| 9 | B | PHENOXYETHANOL | 0.5 | 0.5 |
| 10 | B | CHLORPHENESIN | 0.3 | 0.3 |
| 11 | B | DISODIUM EDTA | 0.1 | 0.1 |
| 12 | B | SODIUM CITRATE | 0.2 | 0.2 |
| 13 | B | SODIUM CHLORIDE | 0.5 | 0.5 |
| 14 | B | CITRIC ACID | 0.05 | 0.05 |
| 15 | C | DIMETHICONE | 3 | 3 |
| 16 | C | DIMETHICONE (and) | 15 | 15 |
| | | DIMETHICONE/VINYL | | |
| | | DIMETHICONE | | |
| | | CROSSPOLYMER | | |
| 17 | C | SILICA SILYLATE | 0.6 | 0.6 |
| 18 | D | POLYMETHYL- | 2 | 2 |
| | | SILSESQUIOXANE | | |
| | | Boron Nitride | | |
| | | Methyl Methacrylate Crosspolymer | 4 | |
| | | (and) Mineral Oil | | |
| | | Nylon-12 Fluorescent Brightener | | 4 |
| | | 230 Salt (and) Polyvinylalcohol | | |
| | | Crosspolymer | | |
| | | Nylon-12 | | |
| | | Diakalyte | | |
| | | Lauroyl Lysine | | |
| | | MICA (and) Titanium Dioxide | | |
| | | Methyl Methacrylate Crosspolymer | | |
| | | HDI/Trimethylol Hexyllactone | | |
| | | Crosspolymer | | |
| | | Freeze/Thaw (−20 C./25 C., | Fail | Fail |
| | | 10 cycles) - Stability | | |
| | | Particle Size (μm) | 5-20 | 5-6 |
| | | Haze (%) | 82.8 | 82.2 |
| | | Transmission (%) | 89.97 | 89.07 |

TABLE 4
Comparative Non-Sunscreen Formulation

| | | | Compar- | Compar- |
|---|---|---|---|---|
| | | | ative | ative |
| | | | Ex 5 | Ex 6 |
| Rank | Phase | INCI US Name | % | % |
| 1 | A | DIMETHICONE | 5 | 5 |
| 2 | A | PEG-9 | 0.9 | 0.9 |
| | | POLYDIMETHYLSILOXY- | | |
| | | ETHYL DIMETHICONE | | |
| 3 | A | DIMETHICONE (and) | 5 | 5 |
| | | DIMETHICONE/PEG-10/15 | | |
| | | CROSSPOLYMER | | |
| 4 | A | PENTAERYTHRITYL | 3 | 3 |
| | | TETRAETHYLHEXANOATE | | |
| 5 | B | WATER (QS) | 49.55 | 49.55 |
| 6 | B | GLYCERIN | 7 | 7 |
| 7 | B | PROPANEDIOL | 3 | 3 |
| 8 | B | CAPRYLYL GLYCOL | 0.3 | 0.3 |
| 9 | B | PHENOXYETHANOL | 0.5 | 0.5 |
| 10 | B | CHLORPHENESIN | 0.3 | 0.3 |
| 11 | B | DISODIUM EDTA | 0.1 | 0.1 |
| 12 | B | SODIUM CITRATE | 0.2 | 0.2 |
| 13 | B | SODIUM CHLORIDE | 0.5 | 0.5 |
| 14 | B | CITRIC ACID | 0.05 | 0.05 |
| 15 | C | DIMETHICONE | 3 | 3 |
| 16 | C | DIMETHICONE (and) | 15 | 15 |
| | | DIMETHICONE/VINYL | | |
| | | DIMETHICONE | | |
| | | CROSSPOLYMER | | |
| 17 | C | SILICA SILYLATE | 0.6 | 0.6 |
| 18 | D | POLYMETHYL- | 2 | 2 |
| | | SILSESQUIOXANE | | |
| | | Boron Nitride | | |
| | | Methyl Methacrylate Crosspolymer | | |
| | | (and) Mineral Oil | | |
| | | Nylon-12 Fluorescent Brightener | | |
| | | 230 Salt (and) Polyvinylalcohol | | |
| | | Crosspolymer | | |
| | | Nylon-12 | 4 | |
| | | Diakalyte | | 4 |
| | | Lauroyl Lysine | | |
| | | MICA (and) Titanium Dioxide | | |
| | | Methyl Methacrylate Crosspolymer | | |
| | | HDI/Trimethylol Hexyllactone | | |
| | | Crosspolymer | | |
| | | Freeze/Thaw (−20 C./25 C., | Fail | Fail |
| | | 10 cycles) - Stability | | |
| | | Particle Size (μm) | 8-12 | 2-4 |
| | | Haze (%) | 84.77 | 66.37 |
| | | Transmission (%) | 90.43 | 90.23 |

TABLE 5
Comparative Non-Sunscreen Formulation

| | | | Compar- | Compar- |
|---|---|---|---|---|
| | | | ative | ative |
| | | | Ex 7 | Ex 8 |
| Rank | Phase | INCI US Name | % | % |
| 1 | A | DIMETHICONE | 5 | 5 |
| 2 | A | PEG-9 | 0.9 | 0.9 |
| | | POLYDIMETHYLSILOXY- | | |
| | | ETHYL DIMETHICONE | | |
| 3 | A | DIMETHICONE (and) | 5 | 5 |
| | | DIMETHICONE/PEG-10/15 | | |
| | | CROSSPOLYMER | | |
| 4 | A | PENTAERYTHRITYL | 3 | 3 |
| | | TETRAETHYLHEXANOATE | | |
| 5 | B | WATER (QS) | 49.55 | 49.55 |
| 6 | B | GLYCERIN | 7 | 7 |
| 7 | B | PROPANEDIOL | 3 | 3 |
| 8 | B | CAPRYLYL GLYCOL | 0.3 | 0.3 |
| 9 | B | PHENOXYETHANOL | 0.5 | 0.5 |
| 10 | B | CHLORPHENESIN | 0.3 | 0.3 |
| 11 | B | DISODIUM EDTA | 0.1 | 0.1 |
| 12 | B | SODIUM CITRATE | 0.2 | 0.2 |
| 13 | B | SODIUM CHLORIDE | 0.5 | 0.5 |
| 14 | B | CITRIC ACID | 0.05 | 0.05 |
| 15 | C | DIMETHICONE | 3 | 3 |
| 16 | C | DIMETHICONE (and) | 15 | 15 |
| | | DIMETHICONE/VINYL | | |
| | | DIMETHICONE | | |
| | | CROSSPOLYMER | | |
| 17 | C | SILICA SILYLATE | 0.6 | 0.6 |
| 18 | D | POLYMETHYL- | 2 | 2 |
| | | SILSESQUIOXANE | | |
| | | Boron Nitride | | |
| | | Methyl Methacrylate Crosspolymer | | |
| | | (and) Mineral Oil | | |
| | | Nylon-12 Fluorescent Brightener | | |
| | | 230 Salt (and) Polyvinylalcohol | | |
| | | Crosspolymer | | |
| | | Nylon-12 | | |
| | | Diakalyte | | |
| | | Lauroyl Lysine | 4 | |
| | | MICA (and) Titanium Dioxide | | 4 |
| | | Methyl Methacrylate Crosspolymer | | |
| | | HDI/Trimethylol Hexyllactone | | |
| | | Crosspolymer | | |
| | | Freeze/Thaw (−20 C./25 C., | Fail | Fail |
| | | 10 cycles) - Stability | | |
| | | Particle Size (μm) | 10-20 | 10-50 |
| | | Haze (%) | 68.47 | 45.43 |
| | | Transmission (%) | 89.87 | 88.27 |

TABLE 6
Inventive Sun-screen Formulation

| | | | Inventive |
|---|---|---|---|
| | | | Ex 3 |
| Rank | Phase | INCI US Name | % |
| 1 | A | BUTYL METHOXYDIBENZOYLMETHANE | 3 |
| 2 | A | HOMOSALATE | 7 |
| 3 | A | ETHYLHEXYL SALICYLATE | 4 |
| 4 | A | OCTOCRYLENE | 4 |
| 5 | A | DIMETHICONE (and) PEG-15/LAURYL | 7 |
| | | POLYDIMETHYLSILOXYETHYL | |
| | | DIMETHICONE CROSSPOLYMER | |
| 6 | A | LAURYL PEG-9 | 1.5 |
| | | POLYDIMETHYLSILOXYETHYL | |
| | | DIMETHICONE | |
| 7 | A | ACRYLATES/STEARYL | 0.8 |
| | | ACRYLATE/DIMETHICONE | |
| | | METHACRYLATE COPOLYMER | |
| 8 | A | DIMETHICONE (and) | 15 |
| | | DIMETHICONE/VINYL | |
| | | DIMETHICONE CROSSPOLYMER | |
| 9 | B | WATER (QS) | 34.6 |
| 10 | B | HYDROXYETHYLPIPERAZINE ETHANE | 1 |
| | | SULFONIC ACID | |
| 11 | B | ADENOSINE | 0.1 |
| 12 | B | PHENOXYETHANOL | 0.7 |
| 13 | B | DISODIUM EDTA | 0.1 |
| 14 | B | CHLORPHENESIN | 0.3 |
| 15 | B | CAPRYLYL GLYCOL | 0.5 |
| 16 | B | GLYCERIN | 7 |
| 17 | B | SODIUM HYALURONATE | 0.2 |
| 18 | B | HYDROXYPROPYL | 9 |
| | | TETRAHYDROPYRANTRIOL | |
| 19 | C | FRAGRANCE | 0.2 |
| 20 | D | METHYL METHACRYLATE | 4 |
| | | CROSSPOLYMER | |
| | | Freeze/Thaw (−20 C./25 C., | Pass |
| | | 10 cycles) - Stability | |

TABLE 7
Comparative Sunscreen Formulation

| | | | Compar- |
|---|---|---|---|
| | | | ative |
| | | | Ex 9 |
| Rank | Phase | INCI US Name | % |
| 1 | A | BUTYL METHOXYDIBENZOYLMETHANE | 3 |
| 2 | A | HOMOSALATE | 7 |
| 3 | A | ETHYLHEXYL SALICYLATE | 4 |
| 4 | A | OCTOCRYLENE | 4 |
| 5 | A | DIMETHICONE (and) PEG-15/LAURYL | 7 |
| | | POLYDIMETHYLSILOXYETHYL | |
| | | DIMETHICONE CROSSPOLYMER | |
| 6 | A | LAURYL PEG-9 | 1.5 |
| | | POLYDIMETHYLSILOXYETHYL | |
| | | DIMETHICONE | |
| 7 | A | ACRYLATES/STEARYL | 0.8 |
| | | ACRYLATE/DIMETHICONE | |
| | | METHACRYLATE COPOLYMER | |
| 8 | A | DIMETHICONE (and) | 15 |
| | | DIMETHICONE/VINYL | |
| | | DIMETHICONE CROSSPOLYMER | |
| 9 | B | WATER (QS) | 38.6 |
| 10 | B | HYDROXYETHYLPIPERAZINE ETHANE | 1 |
| | | SULFONIC ACID | |
| 11 | B | ADENOSINE | 0.1 |
| 12 | B | PHENOXYETHANOL | 0.7 |
| 13 | B | DISODIUM EDTA | 0.1 |
| 14 | B | CHLORPHENESIN | 0.3 |
| 15 | B | CAPRYLYL GLYCOL | 0.5 |
| 16 | B | GLYCERIN | 7 |
| 17 | B | SODIUM HYALURONATE | 0.2 |
| 18 | B | HYDROXYPROPYL | 9 |
| | | TETRAHYDROPYRANTRIOL | |
| 19 | C | FRAGRANCE | 0.2 |
| 20 | D | METHYL METHACRYLATE | 0 |
| | | CROSSPOLYMER | |
| | | Freeze/Thaw (−20 C./25 C., | Fail |
| | | 10 cycles) - Stability | |

TABLE 8
Determination of F/T stability in inventive SPF formulations
with or without Silica Silylate (Aerogel)

| Inventive | |
|---|---|
| Composition | F/T Stability |
| Inventive Ex 1 or | YES |
| Inventive Ex 2 | |
| Inventive Ex 1 or | YES |
| Inventive Ex 2 | |
| without Silica Silylate | |
| (Aerogel) | |

Evaluation of Stability

The samples of the inventive and comparative formulations, as described above, were subjected to Freeze/Thaw testing and subsequent evaluation of stability. In particular, the viscosity of each emulsion was examined to the initial preparations of the samples to confirm acceptability of the initial emulsion. Thereafter samples of the emulsion were subjected to conditions of cycles between −20° C. and 20° C. (a cycle includes exposer to each of −20° C. and 20° C., alternating between the two temperatures for a duration of 12 hours at each temperature) for a 10-day period, after which the viscosity and physical features were evaluated. Differences between the initial features and the 10 day features showed whether the emulsion was stable. Samples of the emulsion were also subjected to 10 freeze/thaw cycles.

As shown in the data tables, compositions according to the disclosure that are in the form of water-in-oil emulsions having an oil phase comprising a silicone elastomer gel comprising an emulsifying elastomer, and at least one non-swelling non-emulsifying silicone elastomer and an aqueous phase comprising water and one or more additional components selected from actives and other additives are not stable to F/T cycles except in the presence of stabilizing spherical powders. Particularly good results were obtained with powders selected from Methyl Methacrylate Crosspolymer and HDI/Trimethylol Hexyllactone Crosspolymer, as shown in Table 1. This stability is achieved whether or not any aerogel is present, as shown in Table 1 and 8. Moreover, this stability is achieved, in particular, in compositions that lack swelling non-emulsifying silicone elastomers. Further, as shown in Table 2-5, inventive compositions according to the disclosure that lack spherical powders including but not limited to Methyl Methacrylate Crosspolymer and HDI/Trimethylol Hexyllactone Crosspolymer cannot be stabilized by the inclusion of aerogel and/or aerogel with other cosmetic powders such as powders with non-spherical particulate shape.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A cosmetic composition comprising:
a water-in-oil emulsion comprising an oil phase, the oil phase comprising at least one silicone elastomer gel comprising at least one emulsifying silicone elastomer and at least one non-swelling non-emulsifying silicone elastomer;
at least one stabilizing agent/spherical powder;
water; and
one or more actives or additives,
wherein the composition exhibits freeze thaw stability, and confers a smooth, non-greasy, silky feel when applied to the skin and other keratinous tissues, and imparts to the skin a soft focus or hazy effect and the appearance of an even complexion.

2. A cosmetic composition according to claim 1, the at least one silicone elastomer gel present at a concentration, by weight, from about 1% to about 20% by weight, based on the total weight of the composition, the at least one silicone elastomer gel comprising at least one emulsifying silicone elastomer selected from emulsifying/self-emulsifying polyether silicone elastomers, polyglyceryl silicone elastomers, polyether dimethicone copolymers, and mixtures thereof.

3. A cosmetic composition according to claim 2, the at least one emulsifying silicone elastomer being selected from dimethicone (and) dimethicone/PEG-10/15 crosspolymer, dimethicone (and) dimethicone/polyglycerin-3 crosspolymer, and polyether-modified crosslinked silicone polymers in dimethicone.

4. A cosmetic composition according to claim 3, further comprising at least one co-emulsifier selected from dimethicone (and) PEG-15/lauryl polydimethylsiloxyethyl dimethicone crosspolymer, PEG-10 dimethicone, dimethicone (and) PEG/PPG-18/18 dimethicone, PEG-9 polydimethylsiloxyethyl dimethicone (and) PEG-9 available, and lauryl PEG-9 polydimethylsiloxyethyl dimethicone.

5. A cosmetic composition according to claim 1, the at least one non-swelling non-emulsifying silicone elastomer present at a concentration, by weight, from about 1% to about 20% by weight of the cosmetic composition, the at least one non-swelling non-emulsifying silicone elastomer selected from polyether-modified silicone crosslinked polymers in dimethicone.

6. A cosmetic composition according to claim 5, the at least one non-swelling non-emulsifying silicone elastomer selected from dimethicone (and) dimethicone/vinyl dimethicone crosspolymer, methyl trimethicone (and) dimethicone/vinyl dimethicone crosspolymer, and diphenylsiloxy phenyl trimethicone (and) dimethicone/phenyl vinyl dimethicone crosspolymer.

7. A cosmetic composition according to claim 6, comprising at least two non-swelling non-emulsifying silicone elastomers.

8. A cosmetic composition according to claim 1, the at least one stabilizing agent/spherical powder present at a concentration, by weight, from about 1% to about 15% by weight of the cosmetic composition, the spherical powder selected from polymer based microspheres, plastic microspheres, that include, methyl methacrylate crosspolymer, HDI/trimethyl hexyllactone crosspolymer, Ethylene/Methacrylate Copolymer, polylactic acid, polymethylsilsesquioxane, polymethylmethylacrlyate, methylmethacrylate crosspolymer, ethylene, acrylic acid copyolymer, aluminimum chlorohudrate, polyethylene, acrylates/ethylhexyl acrylate crosspolymer (and) sodiumpolyacrylate, polylactic acid (and) polyglyceryl-5 laurate, and combinations thereof.

9. A cosmetic composition according to claim 8, the at least one stabilizing agent/spherical powder selected from methyl methacrylate crosspolymer, HDI/trimethyl hexyllactone crosspolymer, and Ethylene/Methacrylate Copolymer.

10. A cosmetic composition according to claim 1, including at least one aerogel present at a concentration, by weight, from about 0.1% to about 5% by weight of the cosmetic composition, the aerogel comprising silica silylate.

11. A cosmetic composition according to claim 1, comprising water present at a concentration, by weight, from about 20% to about 65% based upon the weight of the cosmetic composition.

12. A cosmetic composition according to claim 1, comprising at least one active present in the composition at a concentration, by weight, from about 0.01% to about 80% by weight based on the weight of the composition, the active selected from UV protectant actives, active ingredients for artificially tanning and/or browning the skin (self-tanning agents), dihydroxyacetone (DHA), anti-aging and/or anti-oxidation agents, free-radical scavengers, keratolytic agents, vitamins, anti-elastase agents, anti-collagenase agents, peptides, fatty acid derivatives, steroids, trace elements, extracts of algae, extracts of planktons, enzymes, coenzymes, flavonoids, ceramides, hydroxy acids, enhancing agents, and mixtures thereof.

13. A cosmetic composition according to claim 1, wherein the active is selected from UV protectant actives.

14. A cosmetic composition according to claim 1, comprising one or more additives selected from one or more of an emollient, a hydrating agent, a preservative, a filler, an SPF booster.

15. A cosmetic composition according to claim 1, further comprising a water phase, the water phase comprising water and one or more additional components selected from water soluble actives.

16. A cosmetic composition comprising:
an oil phase and a water phase, the oil phase present at from about 10% to about 50% by weight of the weight of the composition, the oil phase comprising at least one silicone elastomer gel comprising at least one emulsifying silicone elastomer and at least one non-swelling non-emulsifying silicone elastomer, the water phase present at from about 50% to about 90% by weight of the weight of the composition, the water phase comprising water;
at least one stabilizing spherical powder; and
one or more of the oil and the water phase comprising one or more additional components selected from actives and other additives,
wherein the composition exhibits freeze thaw stability, and confers a smooth, non-greasy, silky feel when applied to the skin and other keratinous tissues, and imparts to the skin a soft focus or hazy effect and the appearance of an even complexion.

17. A cosmetic composition according to claim 16:
the at least one silicone elastomer gel comprising at least one emulsifying silicone elastomer selected from dimethicone (and) dimethicone/PEG-10/15 crosspolymer, dimethicone (and) dimethicone/polyglycerin-3 crosspolymer, polyether-modified crosslinked silicone polymers in dimethicone;
the at least one non-swelling non-emulsifying silicone elastomer selected from dimethicone (and) dimethicone/vinyl dimethicone crosspolymer, dimethicone (and) dimethicone/vinyl dimethicone crosspolymer, methyl trimethicone (and) dimethicone/vinyl dimethicone crosspolymer, and diphenyl siloxy phenyl trimethicone (and) dimethicone/phenyl vinyl dimethicone crosspolymer; and
the at least one stabilizing agent/spherical powder selected from methyl methacrylate crosspolymer, and HDI/trimethyl hexyllactone crosspolymer, Ethylene/Methacrylate Copolymer.

18. A cosmetic composition according to claim 17, further comprising at least one co-emulsifier selected from dimethicone (and) PEG-15/lauryl polydimethylsiloxyethyl dimethicone crosspolymer, PEG-10 dimethicone, dimethicone (and) PEG/PPG-18/18 dimethicone, PEG-9 polydimethylsiloxyethyl dimethicone (and) PEG-9 available, and lauryl PEG-9 polydimethylsiloxyethyl dimethicone.

19. A cosmetic composition according to claim 17, wherein the composition is essentially free of one or more of an aerogel, a swelling emulsifying silicone elastomer, a swelling non-emulsifying silicone elastomer, and a swelling agent.

20. A cosmetic composition comprising:
at least one stabilizing spherical powder present at from about about 3% to about 10% by weight of the weight of the composition;
at least one silicone elastomer gel comprising at least one of a water-in-oil (silicone) emulsifying elastomer present at from about 5% to about 10% by weight of the weight of the composition;
at least one of a non-swelling non-emulsifying silicone elastomer present at from about 7% to about 15% by weight of the weight of the composition; and
one or more additional components selected from actives and other additives,
wherein the composition exhibits freeze thaw stability, and confers a smooth, non-greasy, silky feel when applied to the skin and other keratinous tissues, and imparts to the skin a soft focus or hazy effect and the appearance of an even complexion.

21. A cosmetic composition according to claim 20:
the at least one silicone elastomer gel comprising at least one emulsifying silicone elastomer; the emulsifying silicone elastomer selected from dimethicone (and) dimethicone/PEG-10/15 crosspolymer, dimethicone (and) dimethicone/polyglycerin-3 crosspolymer, polyether-modified crosslinked silicone polymers in dimethicone, dimethicone (and) PEG-15/lauryl polydimethylsiloxyethyl dimethicone crosspolymer;
the at least one non-swelling non-emulsifying silicone elastomer selected from dimethicone (and) dimethicone/vinyl dimethicone crosspolymer, dimethicone (and) dimethicone/vinyl dimethicone crosspolymer, methyl trimethicone (and) dimethicone/vinyl dimethicone crosspolymer, and diphenyl siloxy phenyl trimethicone (and) dimethicone/phenyl vinyl dimethicone crosspolymer; and
the at least one stabilizing agent/spherical powder selected from methyl methacrylate crosspolymer, and HDI/trimethyl hexyllactone crosspolymer, Ethylene/Methacrylate Copolymer.

22. A cosmetic composition according to claim 21, further comprising at least one co-emulsifier selected from PEG-10 dimethicone, dimethicone (and) PEG/PPG-18/18 dimethicone, PEG-9 polydimethylsiloxyethyl dimethicone (and) PEG-9 available, and lauryl PEG-9 polydimethylsiloxyethyl dimethicone.

23. A cosmetic composition according to claim 21, wherein the composition is essentially free of one or more of an aerogel, a swelling emulsifying silicone elastomer, a swelling non-emulsifying silicone elastomer, and a swelling agent.
